(19) United States
(12) Patent Application Publication
(10) Pub. No.: US 2010/0151491 A1
(43) Pub. Date: Jun. 17, 2010

(54) CHEMICAL SURFACE NANOPATTERNS TO INCREASE ACTIVITY OF SURFACE-IMMOBILIZED BIOMOLECULES

(75) Inventors: Michael Himmelhaus, Chuo-ku (JP); Sivashankar Krishnamoorthy, Chuo-ku (JP)

Correspondence Address: SUGHRUE MION, PLLC, 2100 PENNSYLVANIA AVENUE, N.W., SUITE 800, WASHINGTON, DC, 20037, US

(73) Assignee: FUJIREBIO INC., Chuo-ku, Tokyo (JP)

(21) Appl. No.: 12/600,638

(22) PCT Filed: May 19, 2008
(86) PCT No.: PCT/JP2008/059596

§ 371 Date: Nov. 17, 2009

Related U.S. Application Data
(60) Provisional application No. 60/938,782, filed on May 18, 2007.

(51) Int. Cl. G01N 33/543; B32B 3/10; C12N 5/00

(52) U.S. Cl. 435/7.2; 436/518; 428/195.1; 435/395

(57) ABSTRACT

The present invention has been achieved in order to solve the problems which may occur in the nanopatterning of biomolecules with an aim to improve the activity and bio-recognition properties of the surface-immobilized biomolecules. A structure for bio-detection according to one aspect of the present invention comprises a large-scale chemical nanopattern of fouling and non-fouling areas fabricated on a homogeneous surface of the structure; and a biomolecule confined to the fouling area.

This application is based upon and claims the benefit of priority from the prior U.S. Provisional Patent Application No. 60/938,782 filed on May 18, 2007; the entire contents of which are incorporated herein by reference.

### TECHNICAL FIELD

The present invention relates to a technology for bio-recognition surfaces.

### BACKGROUND ART

There has been long-standing interest in the patterning of biomolecules, such as antibodies and other proteins, nucleotides and DNA fragments, on surface, mainly with an aim to integrate biomolecules into miniature biological-electronic devices and—in particular with respect to biomolecular sensing—to generate complex biofunctional interfaces with high parallel detection capability (see, for example, A. S. Blawas & W. M. Reichert, Biomaterials, Vol. 19, pp. 595-609, 1998 and references therein).

Among the patterning techniques available, patterning of biomolecules on sub-micron scale, i.e. “nanopatterning”, offers the potential to create complex biofunctional interfaces with a structure and length scale matching that of native biological systems, such as microorganisms, cells, proteins, and nucleotides. Accordingly, high expectation has been raised in view of the impact of nanopatterning on complex biotechnological developments, such as growth of cells and microorganisms, tissue engineering, implant technology, and with respect to multiplexed biosensing in array formats (see, for example, K. L. Christman et al., Soft Matter, Vol. 2, pp. 928-939, 2006 and references therein; P. Mendes et al., Nanoscale Research Letters, Vol. 2, pp. 373-384, 2007 and references therein).

Besides the high structure density achievable, nanopatterning offers the advantage to create patterns of same dimension, or few multiples thereof, of the biomolecules to be surface-immobilized. Accordingly, attempts have been made to influence the adsorption behavior, orientation, and activity of biomolecules by such means. In the following, the work in the field most relevant to the present invention will be briefly summarized.

Valsesia et al. (Langmuir, Vol. 22, pp. 1763-1767, 2006) have prepared nanopatterns consisting of circular patches of self-assembled monolayers (SAMs) of mercaptohexadecanoic acid (MHA) of ˜100 nm diameter embedded in a matrix of hexadecanethiol (HDT). The patterns were derived using self-assembled polystyrene microparticles as masks to structure the underlying homogenous MHA SAM. Enzyme-linked immunosorbent assay (ELISA) measurements obtained from these surfaces were shown to give a 4 times higher signal as compared to the signal of the non-structured counterparts. The authors have concluded that the BSA preferably gets adsorbed on the MHA patches from the height distributions derived from atomic force microscopy (AFM) measurements. The authors have further indicated that the BSA is an ellipsoidal molecule, while having failed to take this fact into consideration to account for the observed height distributions as due to differences in molecular orientation within and outside of the patches.

Cai et al. (Y. Cai and B. M. Ocko, Langmuir, Vol. 21, pp. 9274-9279, 2005) have used self-assembled polystyrene colloidal beads of 300 nm diameter as masks to derive a patterned self-assembled monolayer consisting of ˜60 or ˜120 nm diameter patches of carboxylic acid terminated SAM in a poly(ethyleneglycol) (PEG) matrix on silicon surface. The authors have shown that lysozyme adsorbs selectively within —COOH containing regions. By use of a polyclonal lysozyme antibody, the authors found that the nanopatterned lysozyme maintains its bioactivity. However, the authors do not report about any increase in bioactivity due to patterning as compared to a non-patterned sample.

Agheli et al. (Nano Lett., Vol. 6, pp. 1165-1171, 2006) have prepared gold nano-domes on a silicon surface by means of colloidal lithography. They adsorbed 100 nm polystyrene colloidal beads in a random fashion on the silicon wafer to structure an underlying gold layer by argon ion milling. Poly L-Lysine-g-Polyethylene glycol (PLL-g-PEG) layers were then adsorbed on this composite surface. The authors report that the thickness of the PLL-g-PEG on the gold domes is about 80% that of the bare silicon oxide surface. Then, laminin protein was adsorbed on the surface, which preferentially adsorbed on the gold domes due to a weaker binding of the PLL-g-PEG to the domes. The authors assume that on the gold domes, the PLL-g-PEG layer is completely replaced by the adsorbed laminin. This assumption, however, remains unproven in the article. The bioactivity of the laminin was then tested by means of a polyclonal and a monoclonal anti-laminin antibody. While the monoclonal antibody, that addressed specifically the IKVAV site of the laminin, showed only a weak signal, the polyclonal antibody exhibited significant binding to the nanopatterned laminin. The authors used AFM and quartz crystal microbalance (QCM) measurements for the study of the system. By means of the QCM measurements they observed a higher bioactivity of the nano-patterned laminin with respect to the binding of the polyclonal Ab compared with laminin adsorbed on a non-patterned gold-coated silicon wafer. The authors explain this higher activity with the three-dimensional character of the gold domes, which allow the spill-out of the proteins over the protein-rejecting PLL-g-PEG layer and thus a reduction of steric effects that might hinder specific binding.

Valsesia et al. (Advanced Functional Materials, Vol. 16, pp. 1242-1246, 2006) have shown formation of domes of poly acrylic acid (PAA) in a matrix of polyethylene glycol by combining self-assembly of colloidal beads with plasma-deposition techniques. The authors have further used confocal microscopy analysis to show that the chemisorption of fluorescent protein occurs preferentially on the PAA structures. Experiments on the activity of such patterned proteins have not been performed.

Wadu-Mesthrige et al. (K. Wadu-Mesthrige et al., Biophysical Journal, Vol. 80, pp. 1891-1899, 2001) prepared protein- and antibody nanopatterns by combining self-assembled monolayer technology and AFM-based nanolithography. The AFM was used to create nanoholes in a previously homogenous self-assembled monolayer under liquid conditions, where the liquid contained a second moiety, which then immediately adsorbed in the nanoholes formed by scratching the surface with the AFM tip. The second moiety further comprised a tail group that allowed for selective immobilization of proteins or antibodies. In contrast to the present invention, the authors observed immobilization of proteins and in particular antibodies on the entire surface, not only the nanopatches formed. Therefore, they introduced a washing step that removed the physisorbed molecules from the matrix, while the antibodies chemisorbed on the nanopatches remained (cf. p. 1892 of said article). According to this difference to the present invention, the authors observed a lower density and in particular a lower height of the antibody/antigen complexes on the nanopatches as compared to the findings of the present invention due to the lack of confinement (see for example FIG. 5 of said article and FIG. 4 of the present invention).

The ability of PEG derivatives to resist the adsorption of proteins has been an intense field of research (J. M. Harris, Ed., “Poly(ethylene glycol) chemistry: biotechnical and biomedical applications”, Plenum Press: New York, 1992, pp 199-220).

Of particular interest for applications in nanopatterning as utilized, e.g., in the present invention, are PEG derivatives with the potential to form self-assembled monolayers (PEG-SAMs) on suitable substrates (C. Pale-Grosdemange, J. Am. Chem. Soc., Vol. 113, pp. 12-20, 1991; Kingshott, P.; Griesser, H. J. Curr. Opin. Solid State Mater. Sci. 1999, 4, 403-412; Leckband, D.; Sheth, S.; Halperin, A. J. Biomater. Sci.-Polym. Ed. 1999, 10, 1125-1147; Mona, M. J. Biomater. Sci.-Polym. Ed. 2000, 11, 547-569; J. C. Love et al., Chemical Reviews, Vol. 105, pp. 1103-1169, 2005 and references therein).

Due to their high protein- and cell-resistance, PEG-SAMs have been successfully applied to patterning of proteins, antibodies, cells, and microorganisms on surface (G. P. Lopez et al., J. Am. Chem. Soc., Vol. 115, pp. 10774-10781, 1993; R. S. Kane et al., Biomaterials, Vol. 20, pp. 2363-2376, 1999; S. W. Howell et al., Langmuir, Vol. 19, pp. 436-439, 2003; M. Mrksich et al., Exp. Cell Research, Vol. 235, pp. 305-313, 1997; B. Rowan et al., Langmuir, Vol. 18, pp. 9914-9917, 2002; S. Rozhok et al., Langmuir, Vol. 22, pp. 11251-11254, 2006). Howell et al. reported that targeted bacteria had a higher binding selectivity to complementary antibody patterns than to unfunctionalized regions of the substrate. A comparison to non-patterned surfaces decorated with the same complementary antibody, however, was not performed. Altogether, an increase in activity due to patterning of surface-immobilized biomolecules, cells, or microorganisms over that observed on non-patterned surfaces of same chemistry as the fouling patches of the pattern has not been reported so far in the literature.

### DISCLOSURE OF INVENTION

The present invention has been achieved in order to solve the problems which may occur in the related arts mentioned above.

A structure for bio-detection according to one aspect of the present invention comprising: a large-scale chemical nanopattern of fouling and non-fouling areas fabricated on a homogeneous surface of the structure; and a biomolecule confined to the fouling area.

A structure for bio-detection according to one aspect of the present invention comprising: a chemical nanopattern of fouling and non-fouling areas fabricated on a homogeneous surface of the structure; and an antibody confined to the fouling area.

A structure for bio-detection according to one aspect of the present invention comprising: a large-scale chemical nanopattern of fouling and non-fouling areas fabricated on a homogeneous metal surface of the structure; and a biomolecule confined to the fouling area.

### BRIEF DESCRIPTION OF DRAWINGS

FIG. 1: A method for preparing large-scale nanopatterns, which are homogeneous over a wide area;

FIG. 2: (a) on-chip biosensor substrate bearing a nanopatterning according to the present invention; (b) on-chip multiplex biosensor substrate bearing micropatterned regions (8) that contain nanopatterns according to the present invention;

FIG. 3: (a) Scanning electron microscopy (SEM) image of a colloidal mask deposited on a gold substrate corresponding to step (d) of FIG. 1 of the present invention; (b-d) AFM friction force images of MHA nanopatterns formed on a clean gold surface after reactive ion etching (RIE) (according to step (e) of FIG. 1) and subsequent removal of the colloidal mask.

FIG. 4: AFM tapping mode (TM) and friction force (FF) images of the nanopatterns before and after biomolecule adsorption: a) TM image of MHA patches embedded into the PEG-SH-SAM prior to biomolecule adsorption; b) TM image of the surface shown in (a) after exposure to the sequence of biomolecules (α-MIgG-BSA-MIgG); c) FF image of MHA patches embedded into the CH₃-SAM prior to biomolecule adsorption; d) TM image of the surface shown in (c) after exposure to the same sequence of biomolecules as in (b); Bottom: height distributions (a′), (b′), and (d′) of the line scans indicated in the corresponding images (a), (b), and (d), respectively.

FIG. 5: (upper half) XPS spectra showing the C1S and O1s regions and (lower half) infrared reflection absorption spectroscopy (IRRAS) data showing the CH and the COC stretching regions of the homogenous and nanopatterned SAM indicated in the legend. The samples indicated as RIE 30/60 s+PEG were first coated with MHA, then treated in RIE plasma for 30 s or 60 s, respectively, and subsequently immersed into the PEG solution;

FIG. 6: Schematic presenting the principle of detection of binding events on surface using the technique of surface plasmon resonance (SPR);

FIG. 7: Schematic showing the antibody adsorption on surface through physisorption and chemisorption protocols;

FIG. 8: Biacore sensorgrams of PEG and MHA reference chips showing the responses corresponding to the addition of (I) anti-mouse IgG, (II) BSA and (III) Mouse IgG. The sensorgram on the PEG reference chip shows excellent protein resistance as shown by lack of any response at all;

FIG. 9: SPR responses corresponding to the three consecutive steps of the immunoreaction experiment, performed on different homogenous and nanopatterned Au surfaces. The curves are labeled by the respective surface (the nomenclature for patterns is “nanopatch/matrix”, e.g. MHA/HDT means MHA-SAM nanopatches embedded into a HDT-SAM matrix and accordingly for other types of patterns);

FIG. 10: Histogram comparing the antigen binding capacity (ABC) of homogenous as well as nanopatterned SAM. The bars indicate the statistical errors of the sample-to-sample variation. The results of a total of 26 experiments are shown;

FIG. 11: Analysis of an experiment using colloidal beads with a nominal diameter of 200 nm as etch mask; (a) SPR response to the three subsequent steps of anti-mouse IgG adsorption, BSA passivation (1%), and specific binding of mouse IgG to the anti-mouse IgG; (b/c) AFM topography images of a MHA/PEG-SH SAM nanopattern formed on a SPR chip prior (b) and after (c) adsorption of the antibody/antigen complexes; the line scan at the bottom of the respective image indicates the height profile along the dashed line indicated in the image.

FIG. 12: IRRA spectra of MHA, PEG reference samples and randomly mixed MHA/PEG SAMs.

### BEST MODE FOR CARRYING OUT THE INVENTION

Exemplary embodiments relating to the present invention will be explained in detail below with reference to the accompanying drawings.

Definition of Terms

Nanopattern: A nanopattern is a structure with sizes of the individual features composing the structure below 1 μm. A nanopattern is characterized by its feature size and by the size of its total lateral extension. Both feature size and lateral extension depend in general on the method of fabrication of the nanopattern.

Large-scale nanopattern: A nanopattern with a total extension sufficiently large to allow the study of the nanopattern by means of analytical methods that do not have microscopic resolution, i.e. a resolution of the same dimension as the feature size of the nanopattern. Such non-microscopic methods may be, but are not limited to: surface plasmon resonance, quartz microbalance, acoustic wave sensors, ellipsometry, reflectometry, infrared spectroscopy, nonlinear optical spectroscopy, X-ray photoelectron spectroscopy, impedance spectroscopy, surface potential measurements, contact angle measurements, electrochemical surface measurements, and other surface-analytical tools. In addition to being sufficiently large to match the footprint of the respective non-microscopic method, a large-scale nanopattern also must exhibit sufficient homogeneity across its extension. Thereby, “sufficient homogeneity” means that slight variations in the structures, e.g. in terms of their density, should be on a length scale below the lateral resolution limit of the method applied. Then, the method will simply measure an average value across its footprint on the nanopattern.

Fouling and non-fouling surface: A fouling surface is a surface that shows adsorption of biomolecules when exposed to a solution containing biomolecules in a close to natural state, e.g. under physiological conditions. Instead, a non-fouling surface inhibits such adsorption under these conditions. In most cases, a surface coating is used to render a fouling surface into a non-fouling one. Such surface coatings may be either biomolecules or biopolymers, such as BSA, which inhibit further adsorption of other biomolecules, or organic materials, such as polyethylene glycol). Then, the degree of inhibition of fouling can be quantified by determining the ratio of the amount of adsorbed biomolecules with coating to that without coating. Then, a coating (or the coated surface) is often called “non-fouling”, if the amount of adsorbed biomolecules is reduced by >90% as compared to the non-coated surface.

Homogeneous substrate: A homogeneous substrate is a substrate, which consists either of a single substance or otherwise is homogeneous down to the length scale of the nanoscale features. For example, a polycrystalline metal alloy, which consists of a mixture of crystallites of two different metals, is a homogeneous substrate as long as the length scale of the heterogeneity is smaller than the length scale of the nanopattern built on the surface of the substrate. Typically, the crystallites of such a metal alloy have few nanometers in diameter and they are randomly mixed, so that this condition will be fulfilled in general. A substrate may consist of layers of materials with the topmost layer bearing the nanopattern. Then, the substrate is called homogeneous, if at least the top-layer, e.g. a thin metal film, is homogeneous in the sense defined above.

Chemical pattern: A chemical pattern is a pattern formed by modification of the physico-chemical properties of a surface of the substrate. Such modification may be, but is not limited to, local changes in the wetting properties of the surface, changes in the polarity of the surface, in chemical reactivity, electrical conductivity and resistance, and/or changes in the optical properties of the surface. For example, organic molecules can be adsorbed on the surface of a homogeneous substrate. Different molecules are placed on different areas of the surface, thus changing the surface chemistry of that area according to their own properties. A chemical pattern of organic molecules with fouling and non-fouling properties can be used to prepare a fouling/non-fouling pattern on the surface of the homogeneous substrate.

Continuous metal surface: A continuous metal surface is a closed surface, i.e. a surface exhibiting only pinhole defects of few nanometers in diameter. In particular, a continuous metal surface is conductive and it allows—in principle—the excitation of surface plasmons, i.e. it allows the excitation of surface plasmons when used under conditions that should allow for such excitation.

Antigen binding capacity of the surface: Antigen binding capacity of the surface is expressed as the following:

$$Antigen binding capacity = \frac{Antigen response}{Antibody response} \times 100 ( in \% )$$

### ABBREVIATIONS

ABC—Antigen binding capacity

SAM—Self-assembled monolayer (of organic molecules)

MHA—Mercaptohexadecanoic acid

HDT—Hexadecanethiol

PEG—Polyethylene glycol

PEG-SH—Mercapto polyethylene glycol

MHA/PEG—Patterned surfaces with MHA islands in PEG matrix

MHA/HDT—Patterned surfaces with MHA islands in HDT matrix

EDC—1-ethyl 3-(3-(dimethylamino)propyl)carbodiimide

NHS—N-hydroxy succinimide

PS—Polystyrene

RIE—Reactive ion etching

AFM—Atomic force microscopy

SEM—Scanning electron microscopy

IRRAS—Infrared reflection absorption spectroscopy

XPS—X-ray photoelectron spectroscopy

SPR—Surface plasmon resonance

NSL—Nanosphere lithography

Basic Concepts

One of the key targets in the further development of label-free techniques utilized for biosensing is the optimization of the activity of the biological probe used for targeting the wanted analyte. This is a major issue, because most techniques rely on immobilization of the probe onto a surface interfacing between the specific recognition event and a physical transducer mechanism. This surface confinement of the probe, however, restricts its activity as compared to its native state in liquid due to a number of constraints, like reduced accessibility, steric hindrance, and probe-surface interactions causing degeneration or the blocking of active sites (S. V. Rao et al., Mikrochim. Acta, 128, 127, 1998). Therefore, strategies for immobilization of probes in a close to natural state have been explored intensively during the last decade (S. Chen et al., Langmuir, 19, 2859, 2003). Besides various attempts for oriented probe adsorption that assures an optimum orientation of the active site with respect to the confining surface, nanopatterning could contribute to increase in accessibility and function of the probe by suitable tailoring its immediate environment. Such tailoring could prove promising when the produced patterns are of the dimensions of the order of the probe, thereby allowing for fine adjustments of structure and topography on the relevant scale.

Despite its importance, the investigation of the effect of nanopatterning on probe activity is not a straightforward task. Direct comparison of the nanopatterned substrates with non patterned counterparts using the existing biosensing techniques requires that the nanopatterns are spread over a large area with reasonable homogeneity and integrity. This would enable their study using state-of-the-art systems with sensing areas typically in the range from several hundred microns to several millimeters. Microscopic techniques, such as scanning probe microscopy, which provides lateral resolution on the required nanometric scale—unless carried out under liquid—mainly speak of the mechanical properties of the surface-bound species, such as topography and elasticity. Accordingly, information on structure and state of the immobilized biomolecules is difficult to extract from such data.

We therefore explored the potential of large-scale nanopatterns, which can be analyzed with surface analytical tools that yield averaged information from large areas of the surface. For this purpose, we utilized nanosphere lithography (NSL), which has become a popular tool as a patterning technique recently because the method is cheap and involves extremely simple procedures compared with other nanopatterning techniques, such as electron-beam lithography or scanning probe-related nanolithography. NSL can also be applied to a wide range of organic and inorganic materials. If combined with selective deposition of organic self-assembled monolayers (SAMs), NSL provides a feasible tool for the bio-functionalization of substrates to create next-generation biosensors or other bio-mimetic devices.

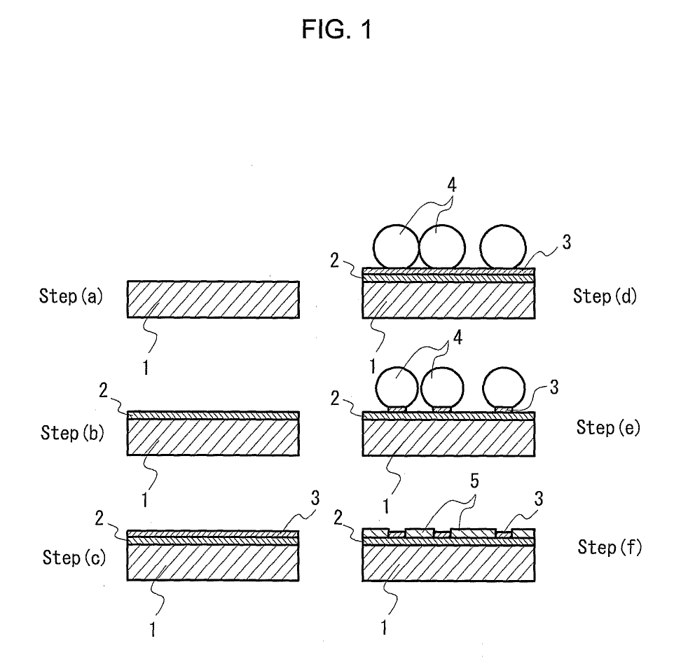

The novel feature of the structure according to the present embodiment can be expressed as a structure for bio-detection through a suitable method, such as surface plasmon resonance, quartz microbalance, surface acoustic waves, ellipsometry, fluorescence labeling, ELISA, or others, where the structure comprises a nanopattern of fouling areas and non-fouling areas fabricated on a surface of the structure, and an antibody confined to the fouling area. For example, the non-fouling area is a non-fouling matrix, the fouling area is a fouling patch embedded in the non-fouling matrix, and the antibody is confined into the fouling patch. The fouling area and the non-fouling area form a nanopattern. Surprisingly, the inventors found that according to this structure, antibody activity is increased as compared to a structure having a non-patterned surface consisting of the fouling area only. For example, the structure, the fouling area and the non-fouling area are corresponding to a silicon or glass substrate 1, patches of fouling SAM (MHA 3) and non-fouling matrix (PEG-SH SAM 5) in FIG. 1 which is later explained, respectively.

The novel feature of the structure according to present embodiment can be expressed differently, namely, as a structure comprising a metal surface, and a large scale nanopattern of fouling areas and non-fouling areas fabricated on a surface of the metal, and a biomolecule confined to the fouling area. For example, the metal surface is a continuous surface (e.g. thin metal film), and the pattern is a large-scale nanopattern which is formed by self-assembly of colloidal particles. Formation of such large-scale pattern of biomolecules on the continuous metal film allows application of highly sensitive label-free methods for detection and analysis of the pattern. Due to the high sensitivity of the methods applicable, an increased antibody activity as compared to a structure having a non-patterned surface became observable.

While the inventors have made the invention in search of a biosensor surface with improved sensitivity, the invention comprises a much broader range of embodiments related to the improved bio-recognition properties of the biomolecule confined to the fouling area of the nanopattern of fouling and non-fouling areas fabricated on a homogeneous surface. Due to this improved bio-recognition, other useful embodiments of the present invention are related to cell growth and cell culture applications, tissue engineering, and the improvement of the biocompatibility of implants. This can be seen as follows.

Most cells are not freely suspended in vivo but adhere to the so-called extracellular matrix (ECM), which is a hierarchically organized three-dimensional organic network with nanoscale structure (P. P. Girard et al., Soft Matter, Vol. 3, pp. 307-326, 2007 and references therein), composed of a collection of insoluble proteins and glycoaminoglycans, in order to function properly, i.e. carry out normal metabolism, proliferation, and differentiation (N. Boudreau & M. Bissell, Current Opinion in Cell Biology, Vol. 10, pp. 640-646, 1998). In addition to maintaining the organization and mechanical properties of tissue, the ECM is also responsible for the generation of specific cell stimuli critical to maintaining cell function and cell response to environmental demands, which are triggered by peptide and carbohydrate ligands of the ECM, which in turn can be recognized by cellular receptors. Accordingly, mimicking the structure and function of the ECM to promote cell growth on artificial surfaces for applications in tissue engineering, neuron guiding, and the development of fully biocompatible implants is one of the main targets of state-of-the-art research in bio-nanotechnology. A well-explored strategy comprises coating of the artificial surface with ECM molecules to mimic the natural host environment of the cells. For example, the ECM glycoprotein fibronectin contains the RGD peptide sequence, which specifically binds to integrin receptors present in the membrane of, e.g., mammalian cells (R. D. Bowditch et al., J. Biolog. Chem., Vol. 269, pp. 10856-10863). The integrins, in turn, are well-known to form focal adhesion points, which play a crucial role in cell signaling and cell adhesion to the ECM (F. G. Giancotti & E. Ruoslahti, Science, Vol. 285, pp. 1028-1032, 1999). Accordingly, fibronectin-coated artificial surfaces have been reported to bind mammalian cells, such as endothelial cells (see for example, M. Mrksich et al., Exp. Cell Res., Vol. 235, pp. 305-313, 1997).

However, as pointed out by Mrksich (M. Mrksich, Chemical Society Reviews, Vol. 29, pp. 267-273, 2000), there are limitations to this concept, because proteins, glycoproteins, or other ECM matrix molecules used as an interface between the artificial surface and the cells may undergo structural changes during the process of surface adsorption and thus lose their natural function. Further, the density of functional receptors on surface remains poorly controllable, since some of the ligands targeting cell receptors may become inactive due to interaction with the surface or due to steric hindrance in an environment strongly confined by the surface. Effects of this kind may cause different cell behavior even when using the same cell adhesion molecule, as reported by Garcia and coworkers (A. J. Garcia et al., Mol. Biol. Cell, Vol. 10, pp. 785-798, 1999), who found a pronounced substrate effect when using fibronectin as the cell-adhesive surface coating on different kinds of polystyrene substrates. More recently, Spatz and coworkers have demonstrated that even minute changes in the density of integrin ligands on the nanometer scale can be decisive for whether a cell adheres or keeps migrating on the corresponding surface (M. Arnold et al., Chem Phys Chem, Vol. 5, pp. 383-388, 2004; E. A. Cavalcanti-Adam et al., European Journal of Cell Biology, Vol. 85, pp. 219-224, 2006).

Therefore, the future development of complex bio-organic surfaces capable of stimulating, controlling, and programming cell adhesion, growth, proliferation, and function for applications in tissue engineering, implant technology, and basic research, such as stem cell studies on the influence of external stimuli to stem cell development and proliferation, depends strongly on the ability to create surfaces with the wanted density, functionality, and activity of cell receptor ligands. The surprising observation subject to the present invention that nanopatterning improves the activity of surface-bound antibodies, and in more general biomolecules on surface, therefore can be directly applied to the improvement of cell-surface interactions. For example, a variety of antibodies targeting specific integrins or specific integrin-subunits are commercially available (for example, Millipore Co., Billerica, USA, currently offers over 150 different anti-integrin antibodies, each of them specific to a different kind of integrin or integrin-subunit). Accordingly, by means of the present invention, nanopatterns with a high density of active integrin ligands may be fabricated, where each ligand may address the targeted cell in a highly specific manner. Then, the influence of density and nature of the ligands as well as their composition on surface on cell adhesion, growth, and function can be studied with ease. It must be noted that the use of antibodies in this context promises to be advantageous over that of the cyclic RGD sequence utilized in state-of-the-art work (see, e.g., M. Arnold et al., E. A. Cavalcanti et al.), because the RGD sequences (linear or cyclic) are limited in the number of potential targets they may bind to as well as in their affinity towards them (M. C. Beckerle, ed., “Cell Adhesion”, B. D. Hames, D. M. Glover, series eds., “Frontiers in Molecular Biology”, Oxford University Press, Oxford, UK, pp. 100ff, 2001; M. Kato & M. Mrksich, Biochemistry, Vol. 43, pp. 2699-2707, 2004, and references therein). Antibodies, however, are available in a much wider range, addressing more targets with higher specificity and selectivity (e.g. also cell surface/trans-membrane proteins different from integrins or those integrins that do not bind to RGD) and with fine-tunable affinity (e.g. by variation of their complementarity determining regions (CDRs)). Therefore, in connection with the present invention, the use of antibodies for stimulating, controlling, and programming cell adhesion, growth, proliferation, and function promises the generation of more complex, more specific and better fine-tuned cell stimuli and thus to pave the way for the development of bioorganic surfaces of much higher complexity than those achievable with state-of-the-art technology.

In the examples below, nanosphere lithography is utilized for nanopattern generation solely because of its ease of preparation and the large scale, on which patterns may be reproducibly fabricated. The latter has been important mainly for the proper characterization of the resulting structures with macroscopic spectroscopic methods, such as X-ray photoelectron spectroscopy (XPS), infrared reflection absorption spectroscopy (IRRAS), and surface plasmon resonance (SPR). However, the findings disclosed in the present invention are compatible with any other method of nanopatterning, such as photolithography (UV, deep-UV, X-ray), particle beam lithography (electrons, atoms), scanning probe lithography (dip-pen lithography, AFM-based lithography), nanografting, micro/nanocontact printing, and others (see, for example, Chrisman et al, Soft Matter, Vol. 2, 928-939, 2006; Mendes et al., Nanoscale Res. Left., Vol. 2, pp. 373-384, 2007, and references therein).

Materials to be Used

Substrate: Except for its homogeneity as defined above, there are no particular requirements on the substrate. Of course, it must be suitable for the chosen approach of (large-scale) nanopatterning and—for sensing applications—it should be compatible with the method used for detection of biomolecular events, such as specific binding events, on the nanopattern. For example, in case of use with a surface plasmon resonance sensor, the substrate should be transparent for the operating wavelength of the sensor and bear a thin metal film, e.g. gold film, on a surface. In case of use with a quartz microbalance, the substrate may comprise a quartz crystal, in the case of use with a FET transistor, it may be the material of the gate of the FET, and so forth.

Further, the substrate may be chosen such that it is compatible with the chosen way of (large-scale) nanofabrication. In the case of using nanosphere lithography of colloidal particles, for example, a substrate that may be rendered highly hydrophilic is advantageous, since hydrophilicity supports the formation of colloidal masks with low defect density.

Another way of choosing the substrate is connected to the chemical pattern of fouling and non-fouling areas on a surface of the substrate. The substrate should provide sufficient adhesion for fouling and non-fouling materials even when nanopatterned and farther be compatible with the chemistry of the adhesion as well as nanopatterning processes.

Formation of nanopatterns: In general, nanopatterns on surfaces can be formed using two different approaches, one of which permits parallel fabrication of features spanning large areas on surface using self-assembly methods or using serial methods of fabrication that can form a desired number of nanoscale features with precise dimensions on precisely chosen area of the surface.

The self-assembly means of fabrication could involve use of colloidal beads (randomly or regularly adsorbed), block copolymer self-assembly in phase-separated thin films, or using block copolymer micelles deposited on the surface, or surface micelles formed by adsorption of copolymer molecules from solution phase through use of dendrimers or phase separation of polymer blends (polymer-demixing).

Large scale parallel fabrication of features could also be achieved without using a self-assembly approach, but through use of rather expensive tools like laser interferometry, X-ray interference lithography and DUV lithography. These latter methods may be combined with nanocontact printing or nanoimprint lithography (see, for example, Christman et al., Soft Matter, Vol. 2, pp. 928-939, 2006 and references therein) to allow fast reproduction of the patterns from a master and accordingly to reduce the costs of the overall pattern fabrication.

The parallel fabrication approaches stated above are best chosen when a precise placement on the surface is not a goal and the primary advantage is a homogenous nanopatterning over a large area.

The serial techniques for nanopatterning may use techniques such as E-beam lithography, Focused ion beam lithography, Dip pen lithography (DPN), Nanoscale dispensing using AFM tips as nanospotters (NADIS), and SPM lithography involving techniques like nanoshaving and Field enhanced oxidation (see also K. L. Christman et al., Soft Matter and P. Mendes et al., Nanoscale Research Letters).

Fouling material: A fouling material, i.e. a material, which absorbs biomolecules close to their native state, e.g. from physiological solution, is in principle not difficult to find, since most organic compounds that can be used for the formation of nanopatterns, such as SAM, polymers or mixtures of polymers (e.g. spin-coated onto a surface), colloids, liquid crystals, and so forth, show fouling to a certain extent. The reason for this is that biomolecules, e.g. proteins, adsorb to essentially all non-natural surfaces (M. Mrksich, Chem. Soc. Rev., Vol. 29, 267-273). Therefore, in fact, the problem is to find a non-fouling material that suppresses biomolecular adhesion as much as possible. The choice of which fouling material should be used can be simply made on basis of other restrictions, such as compatibility with the method used for sensing in case of a sensor application, the way of coupling the biomolecule to the fouling areas (e.g. via a particular chemical linker group or electrostatically, etc.). Also, compatibility with the processes used for large-scale nanopatterning or the formation of the chemical pattern may decide on the material of choice.

Non-fouling material: Non-fouling materials that can be used for application as non-fouling matrix of the present invention are more difficult to identify than the fouling materials. In general, to render a surface protein-resistant it may be coated with a natural biological compound, such as a protein or a different biopolymer, that adsorbs on the fouling areas of the surface and then inhibits adsorption of further biomolecules basically by mimicking a natural environment.

Another strategy is related to the use of chemical substances, such as particularly designed polymers, that inhibit biomolecular adsorption onto their surface. Here, mainly poly(ethylene glycol) (PEG) and oligo(ethylene glycol) (OEG) derivatives have been playing an important role (J. M. Harris, Ed. Poly(ethyleneglycol) chemistry: biotechnical and biomedical applications; Plenum Press, New York, 1992). Those non-fouling molecules may contain certain linker groups to facilitate adsorption of the molecule onto a surface of the substrate. For example, in the case of a gold surface, the molecule may contain a thiol group (K. L. Prime, G. M. Whitesides, J. Am. Chem. Soc., Vol. 115, pp. 10714-1721, 1993; S. Tokumitsu et al., Langmuir, Vol. 18, pp. 8862-8870, 2002). In the case of a semiconductor oxide or metal oxide surface, the molecule may contain a silane or siloxane or phosphate group to allow its adsorption onto a surface of the substrate.

Jeon et al. (J. Colloid. Interface Sci., Vol. 142, pp. 149-158, 1991) have shown that the degree of protein resistance of PEG derivatives is a function of their density. Accordingly, the extent to which a surface can be rendered non-fouling depends on both the molecule and the surface used and on the ability of the molecule to form a dense layer on the surface. A system that shows very high packing density and accordingly high resistance to biomolecular adsorption is described for example in Tokumitsu et al. (cf. above) and Herrwerth et al. (S. Herrwerth, Langmuir, Vol. 19, pp. 1880-1887, 2003). This system is applicable only to metal surfaces. Examples for PEG derivatives on metal oxide and semiconductor surfaces are given for example in the book of J. M. Harris (cf. above) and the article of Leckband et al. (D. Leckband et al., J. Biomater. Sci., Polym. Ed., Vol. 10, pp. 1125-1147, 1999).

According to subtle differences in packing density and structure of the layer formed by the non-fouling molecule on a surface of the substrate, the extent of suppression of biomolecular adsorption may vary according to the description given in the definition of terms. As stated there, a surface coated with a non-fouling molecule is called “non-fouling surface” if it suppresses at least 90% of the biomolecular adsorption found on a non-coated surface of the substrate.

Bio-Functionalization:

The bio-functionalization of the surface may involve physisorption and/or chemisorption protocols to immobilize the probe molecules to the surface. Physisorption protocols rely on non-covalent interactions such as attraction of opposite charges, hydrophobic interactions, hydrogen bonding, or use of specific interactions such as that between avidin & biotin. The physisorption means offers some very useful handles for means of controlling the quantity of protein adsorbed on a surface, and to give them an orientation. Physisorption of the biomolecules could be carried out either directly, or through a monolayer of molecules that are already attached to the surface mediating the immobilization process. Such monolayer of mediator molecules could for instance be self-assembled monolayers (SAM), or ultrathin polymer films such as polyelectrolytes like poly(lysine). Antibody immobilization with the Fc fragment oriented towards the surface could be obtained through a prior immobilization of a mediator protein, such as protein A, protein G, or recombinant protein A-G, that exhibits high affinity for the Fc fragment-of the antibody. There could thus be a combination of different physisorption protocols that can be handled to immobilize the biomolecule of interest, and in a form (activity, orientation, etc.) that is of interest to the application of interest. Alternatively, one would use chemisorption protocols to covalently immobilize the molecules on surface. These protocols frequently use mediator molecules which at their one end form tight bonds with the surface (such as thiol on Au) and expose the other end containing a useful functional group (such as —COOH or —NH₂) to the surface. For instance, a surface consisting of a SAM with —COOH head group can be activated with NHS/EDC reagent to form an activated surface which upon incubation with a protein molecule would readily undergo a condensation reaction with —NH₂ functional groups in the protein to form a peptide bond. This protocol known as amine coupling is widely used to attach biomolecules to the surface. The biomolecule can then be covalently linked to the surface through an appropriate reaction involving the terminal functionality of the underlying SAM. Photoactive functional groups can be introduced to the surface that can enable capture of the biomolecule of interest through photoirradiation at a suitable wavelength. To circumvent the difficulties associated with orienting the whole antibody molecules, researchers have cleaved the molecule to isolate the F(ab) fragments using enzymatic digestion methods. The Fab fragments by the nature of cleavage have a —SH group at one end that enables chemisorption to gold surface and also with an orientation such that the binding sites are exposed at the surface.

### Embodiments

On-Chip Biosensor:

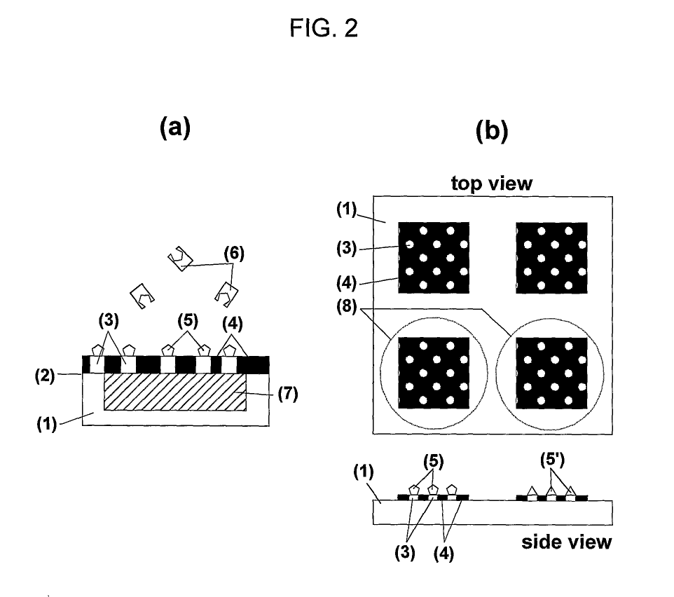

An embodiment of the present invention is related to the detection of an analyte. As shown in FIG. 2a, a substrate (1) having a surface (2) bearing a nanopattern of fouling (3) and non-fouling (4) areas is used to confine a probe molecule (5) to the fouling patches. The probe molecule is used to specifically bind the wanted analyte (6). In one modification of the embodiment, the substrate comprises a transducer mechanism (7) for label-free sensing of the binding event.

Micro-nano arrays for multiplex biosensing: Another embodiment of the present invention is related to the parallel detection of a multitude of different analytes (FIG. 2b). To achieve this a surface of the substrate (1) of the on-chip biosensor as described above is divided into regions (8) in the micrometer to millimeter regime, each region bearing a nanopattern of fouling (3) and non-fouling (4) areas and a probe molecule (5) confined to the fouling area. A multitude of different analytes can then be detected simultaneously by decorating the fouling areas of different regions with a different probe molecule (5′).

Cell adhesion/Cell culturing: One embodiment of the present invention is related to the controlled adhesion of cells onto the nanopattern and their culturing. The nanopattern consistent of fouling and non-fouling areas confining biomolecules that influence cell adhesion to the fouling area is sufficiently large to support adhesion of entire cells. The structure of the nanopattern as well its biofunctionality is tailored such that it promotes the evolution of a wanted property of the adhered cell.

Tissue engineering: In another embodiment of the present invention, the nanopattern described for cell adhesion/cell culturing is tailored such that it promotes the growth of a wanted tissue, for example by influencing stem cells adhered to the pattern such that they evolve into the wanted tissue.

Implant technology: Another embodiment of the present invention uses the nanopattern described in one of the embodiments above as bio-compatible coating of an implant. This may be achieved by tailoring the nanopattern of fouling and non-fouling areas with biomolecules confined into the fouling area in such a way that it promotes biocompatibility of the implant. For example, the nanopattern may facilitate adsorption and/or growth of endogenous biomolecules on the implant, thereby rendering it bio-compatible.

### Examples

### Example 1

### Antibody Nanopatterning on a Gold-Coated Substrate Materials

Chemicals: 16-mercaptohexadecanoic acid (MHA, COOH-SAM) and hexadecanethiol (HDT, CH₃-SAM) were obtained from Sigma-Aldrich Japan K. K., Tokyo, Japan. Mercaptopolyethyleneglycol (PEG) with a molecular weight of ˜2000 g/mol was custom synthesized by Prochimia, Inc., Poland. Chloroform and ethanol (both p.a. grade) were purchased from Wako Pure Chemical Industr. Ltd., Osaka, Japan. Polybead carboxylated polystyrene (PS) microspheres with a mean diameter of 0.454 μm were obtained from Polysciences, Inc., Warrington, Pa. N-hydroxysuccinimide (NHS) and 1-Ethyl-3-[3-dimethylaminopropyl]carbodiimide hydrochloride (EDC) and ethanolamine hydrochloride, 1 M, were obtained from Biacore K. K., Tokyo, Japan, as a part of the amine coupling kit. Phosphate buffered saline (PBS) was obtained in the form of tablets from MP biomedicals. Each tablet was dissolved in 100 mL millipore water to obtain a pH of 7.2. Monoclonal mouse IgG against human α-fetoprotein (MIgG; “antigen”) and polyclonal goat anti-mouse IgG (α-MIgG; “antibody”) were prepared in-house; bovine serum albumine (BSA) was received from Wako.

Substrates: 4′ Si wafers with <100> orientation were obtained from Komatsu Silicon, Miyazaki, Japan, and diced into pieces of required dimensions. The gold coating used for IRRAS measurement was prepared by evaporating 5 nm Cr (Megatech Ltd., Huntington, UK) followed by 30 nm Au (99.99%; Furuuchi Kagaku K.K., Tokyo, Japan) onto a square Si chip of 1.8×1.8 cm² dimensions. The gold-coated glass chips used for the SPR measurements were obtained from Biacore, as a part of the Au SIA kit.

Methods.

Preparation of Chemical Nanopatterns: A method for preparing homogeneous large-scale nanopatterns is show in FIG. 1. In the first step (a), a silicon or glass substrate 1 is prepared. Then, the substrate 1 is coated with a 30-50 nm thick gold film 2 using Cr as adhesion promoter (step (b)). The gold 2 is chemically functionalized by means of MHA 3 (step (c)), then the colloidal mask 4 is deposited as described in the literature (step (d)). The colloidal beads 4 form contact points of about 200 nm in diameter with the MHA-coated substrate 1. The non-covered MHA 3 is removed from the gold surface 2 by means of RIE (step (e)). Finally the colloidal mask 4 is removed and the free gold area is coated with PEG-SH SAM 5 for introduction of a protein-resistant matrix (step (f)).

Characterization of Nanopatterns: IRRA spectra were obtained by means of a JEOL FTIR 680 plus spectrometer, equipped with a high angle reflection unit (80 incidence) and a dry-air purge system. Spectra were referenced against a gold substrate of same origin coated with a perdeuterated alkanethiol. XP spectra were acquired with a JEOL SP-9200 surface analysis system at a base pressure of 5×10⁻⁷ Pa. The non-monochromatic MgKα source was operated at 100 W emission power. The hemispherical electron analyzer was set to a pass energy of 50 eV for wide, and 10 eV for detailed scans. The system was operated in macroscopic detection mode with a footprint of the electron analyzer entrance aperture on the sample surface of about 3 mm diameter. SEM images were collected with a Hitachi S-4200, Hitachi, Inc., Tokyo, Japan. Scanning probe images were acquired with a Digital Instruments Dimension 3100, Nanoscope IV (Veeco Instr., Tokyo, Japan) or a JEOL JSPM-5200 (JEOL, Tokyo, Japan), using ultrasharp silicon nitride coated Si cantilevers with a force constant of 0.12 N/m from Mikromasch S. L., Madrid, Spain.

In-situ Antibody/Antigen Adsorption: The kinetics of antibody adsorption onto the patterned substrates and subsequent binding of the antigen was monitored in-situ by means of a Biacore-X SPR system (Biacore). Degassed Phosphate Buffered Saline (PBS) solution at pH 7.2 was used as the running buffer. The samples were prepared in the same buffer, and were injected after ensuring a stable flow of the running buffer. Flow rates of 20 μl/min and 60 μl sample quantity were used each time, and the experiments were carried out at 25° C. The experiments were performed on Biacore chips consisting of SAM of PEG, MHA and PEG/MHA patterns. SPR response of the following three consequent pulses was monitored: (1) 37 μg/mL solution of α-MIgG, (2) BSA, 1%, and (3) MIgG at 50 μg/mL concentration.

Results and Discussion.

Nanopatterns were formed of SAMs of ω-substituted alkanethiols on a Au surface, with terminal functional groups that either favor or suppress antibody adsorption on the surface. Two different types of nanopatterns were compared, one, which would lead to confinement of the antibody molecules, and another, which would not. The confinement inducing patterns provided fouling nanopatches in a non-fouling background. These patterns consisted of ˜200 nm circular areas of either COOH— or CH₃-SAM embedded into a matrix of a PEG-SH SAM (COOH/PEG or CH₃/PEG). A second type of nanopatterns consisted of COOH-SAM patches in a CH₃-SAM matrix, thereby offering a fouling patch in a fouling background expected to yield no confinement.

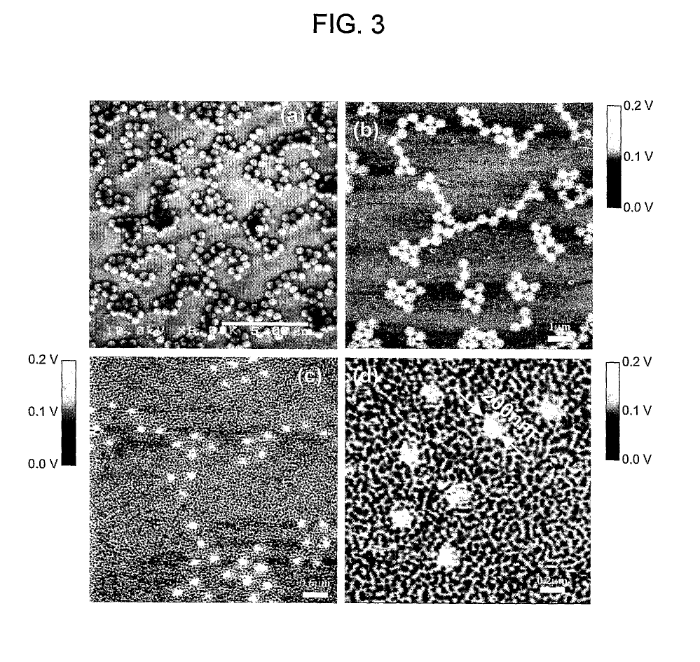

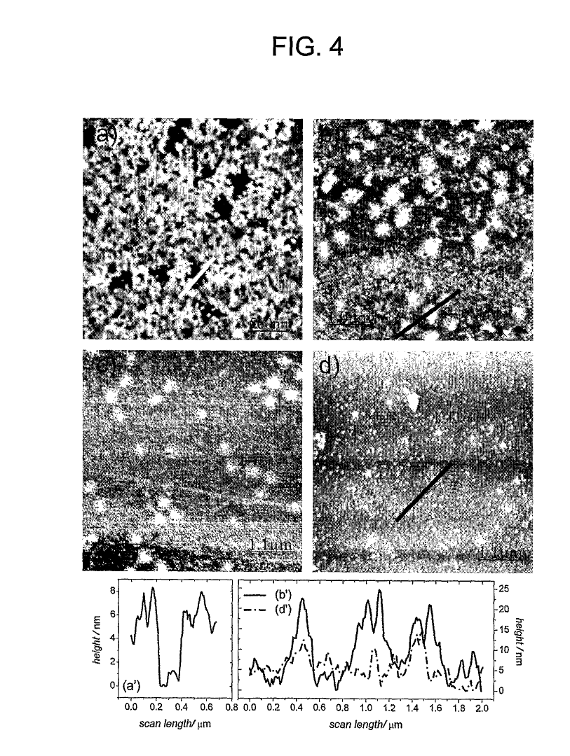

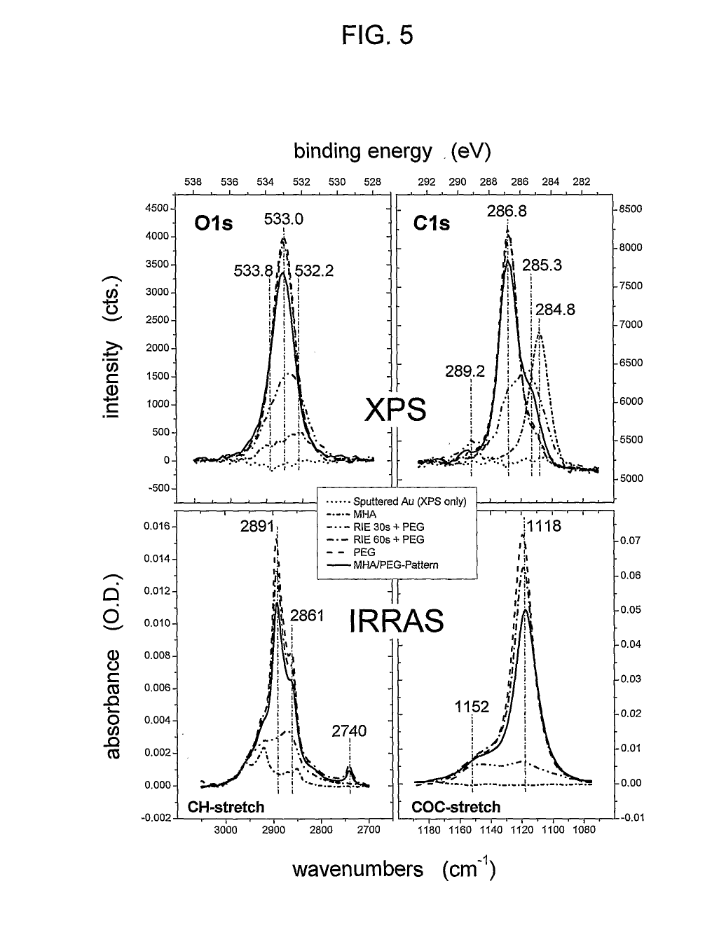

As detailed above (cf. FIG. 1), the binary patterns were fabricated by a combination of molecular self-assembly, nanosphere lithography, and RIE, using 500 nm PS microspheres as etch masks. The characterization of the patterns was carried out using SEM and AFM to assess the dimensions of the nanoscale features. As shown in the SEM image of FIG. 3a, the colloidal etch mask forms a random-close-packed monolayer with a surface coverage of about 54% (M. Himmelhaus and H. Takei, Phys. Chem. Chem. Phys., Vol. 4, pp. 496-506, 2002) on the gold surface pre-functionalized with either mercaptohexadecanoic acid (MHA, COOH-SAM) or hexadecanethiol (HDT, CH₃-SAM). After the RIE process and subsequent removal of the colloidal mask, the remaining SAM features are clearly discernible as circular patches as proven in FIGS. 3b-d (for MHA) and FIG. 4c (for HDT) by friction force mode AFM. Size and distance of closely neighboring features depend on the etching time of the RIE process as exemplified in FIG. 3b-d for MHA patches. While 15 s of RIE still yields interconnected patches, 60 s of RIB produces isolated patches of about 200 nm diameter under the used working conditions. Therefore, for all experiments 60 s of RIE were chosen. After backfilling of the surface with a thiol-terminated polyethylene glycol (PEG), the SAM patches appear as depressions of ˜8 nm depth as shown for MHA in the topological AFM image of FIG. 4a. This observation is in conformity with what could be expected, given that the PEG-SH SAM is 10 nm thick and the COOH-SAM is ˜2 nm. Using Lambert-Beer's law and an attenuation length of the photoelectrons of 35.0 Å at 285 eV, an independent determination of film thickness by means of X-ray photoelectron spectroscopy (XPS) via the attenuation of the Au4f7/2 peak yielded in good agreement 2.1 nm film thickness for the COOH-SAM and 9.6 nm for the PEG-SH SAM. XPS was further used to examine the elemental composition of the surfaces and to determine the surface coverage of the nanoscale MHA patches. FIG. 5 (upper half) displays the O1s and C1s regions after different surface treatments. The peak positions determined via fitting of Voigt profiles to the spectra after performing a Shirley background correction match their respective literature values (cf. e.g. D. A. Hutt & G. J. Leggett, Langmuir, Vol. 13, pp. 2740-2748, 1997). The C1s region reveals the different chemical shifts of aliphatic and carboxylic species. The MHA shows mainly an aliphatic signal at 284.8 eV, while the homogenous PEG film exhibits a strong ether peak at 286.8 eV. The nanopattern, however, shows a clearly observable aliphatic shoulder at 285.3 eV in the ether peak, indicating the proper formation of the MHA/PEG pattern. In the O1s region, the carboxyl group of the MHA reveals two peaks for hydroxyl and carbonyl oxygen at 532.2 eV and 533.8 eV, respectively. The O1s ether peak of the PEG is found at 533.0 eV. The only slight decrease in intensity in the ether peak of the MHA/PEG pattern after 60 s RIE as compared to the homogenous PEG film underlines the success of the nanopatterning process.

The latter drop in intensity was used to calculate the relative surface coverage of the MHA-covered patches on the nanopatterns as compared to the homogenously covered PEG surface (FIG. 5 upper half) according to the relation for the MHA surface fraction, χ_{MHA}=(I⁰_{PEG}−I_{PEG})/(I⁰_{PEG}−I_{MHA}), where I⁰_{PEG} is the intensity of the ether peak on the non-patterned PEG sample, I_{PEG} its intensity on the patterned PEG/_{MHA} sample, and I_{MHA} that of the _{MHA} reference sample. Typically, the relative coverage with MHA patches obtained that way amounts to 8-10%, which is in good agreement with the theoretical estimate assuming a packing density of the colloidal mask of 54% and a contact point diameter of about 200 nm as observed by AFM.

Another important issue related to nanofabrication of fouling patches embedded in a non-fouling matrix is the proof that the patterning does not influence the structure, and thus potentially the non-fouling behavior, of an otherwise non-fouling matrix. We chose IRRAS of patterned and non-patterned surfaces for a comparison of their structure by means of the COC and CH stretching regions as displayed in FIG. 5 (lower half). As was demonstrated for similar PEG systems, (S. Tokumitsu et al., Langmuir, Vol. 18, pp. 8862-8870, 2002) the resonance at 1118 cm⁻¹ can be assigned to a COC stretching mode with a transition dipole moment along the molecular main axis, while the mode at 1152 cm⁻¹ is oriented in perpendicular direction. Therefore, with help of the IRRAS selection rules, which state that a molecular transition dipole moment is only observable in the case that its orientation is not parallel to the metal surface (which follows immediately from the Fresnel laws of reflection applied to the dielectric/metal boundary), the intensity ratio of these two modes can be used for a qualitative interpretation of the structure of the PEG film formed. In the case of an amorphous structure, the intensity ratio should be close to unity, in the case of a more ordered structure with a preferred orientation of the molecules perpendicular to the surface, the 1118 cm⁻¹ mode should dominate. Corresponding considerations hold for the EG CH₂ symmetric stretching modes at 2891 and 2861 cm⁻¹, respectively, although their interpretation is somewhat hampered due to the presence of other modes in this region (S. Tokumitsu et al.). Only the EG combination vibration at 2740 cm⁻¹, which is also parallel to the molecular axis of the PEG, is clearly separated from all other modes and thus the most suitable CH stretching mode to indicate a preferred orientation of the molecules along the surface normal. The duration of RIE exposure for formation of the nanopatches (cf. step (e) in FIG. 1) should be optimal such that the removal of MHA molecules in the non-protected areas is complete and such treated surface is suitable for backfilling with PEG thiol. The duration of the RIE treatment was optimized by exposing a homogenous MHA surface to the oxygen plasma, followed by backfilling with the PEG thiol and monitoring the IRRA and XP spectra for the structure and composition of the PEG layer formed. As shown in FIG. 5, the results indicate that a 60 s RIE exposure was necessary (at the given power and partial pressure of oxygen) to achieve a structure similar to that of the PEG film adsorbed on a fresh gold surface, while a 30 s exposure led to an amorphous matrix most likely due to the incomplete removal of COOH-SAM residues. This becomes in particular evident from the O1s and C1s XP spectra of the 30 s sample, which indicate an unexpected presence of carboxylic and aliphatic species on the surface, respectively. (cf. FIG. 5, upper half). Summarizing, the surface analysis demonstrates that when proper conditions for the process of nanopatterning are chosen, the structure of the PEG matrix on the patterned sample is unaffected by the presence of MHA patches.

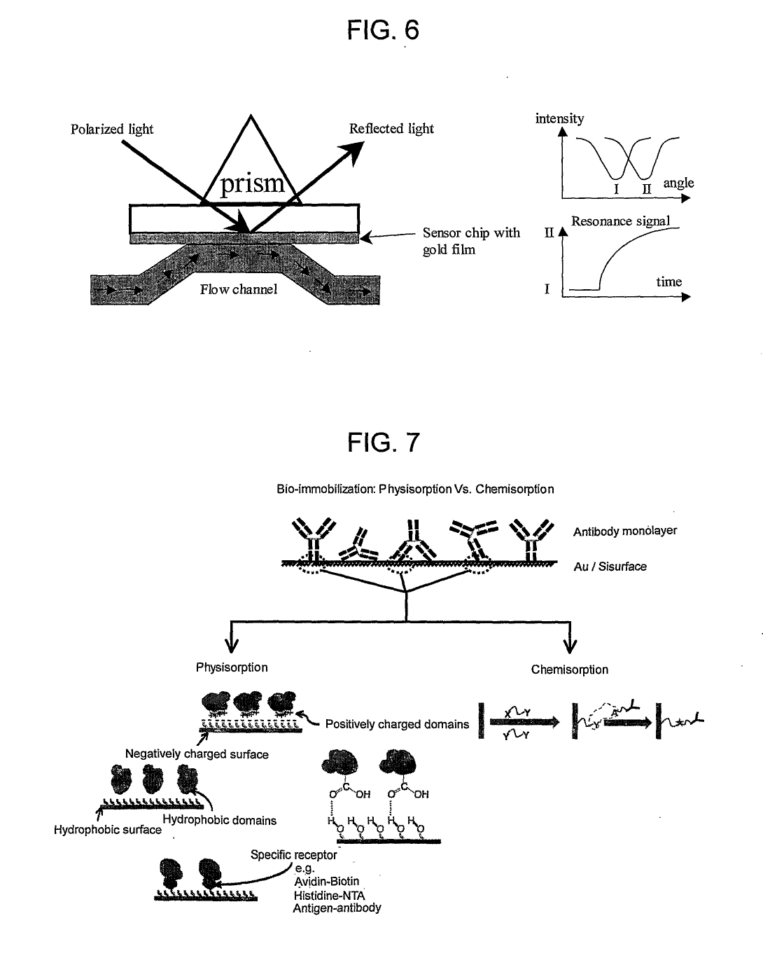

Biomolecular binding events occurring on thus prepared surfaces were monitored using a commercial surface plasmon resonance set-up from Biacore AG (Biacore X) as sketched in FIG. 6 (M. Malmqvist, Biochem. Soc. Trans., Vol. 27, pp. 335f, 1999). The SPR system measures changes in the refractive index in close vicinity of the gold surface, typically within a few hundreds of nanometer distance. Accordingly, the SPR sensorgrams are displayed in RU, i.e. ‘refractive index units’, which—for organic matter—can be approximately converted into a change in mass density on the surface according to the formula: 1000 RU=1 ng/mm².

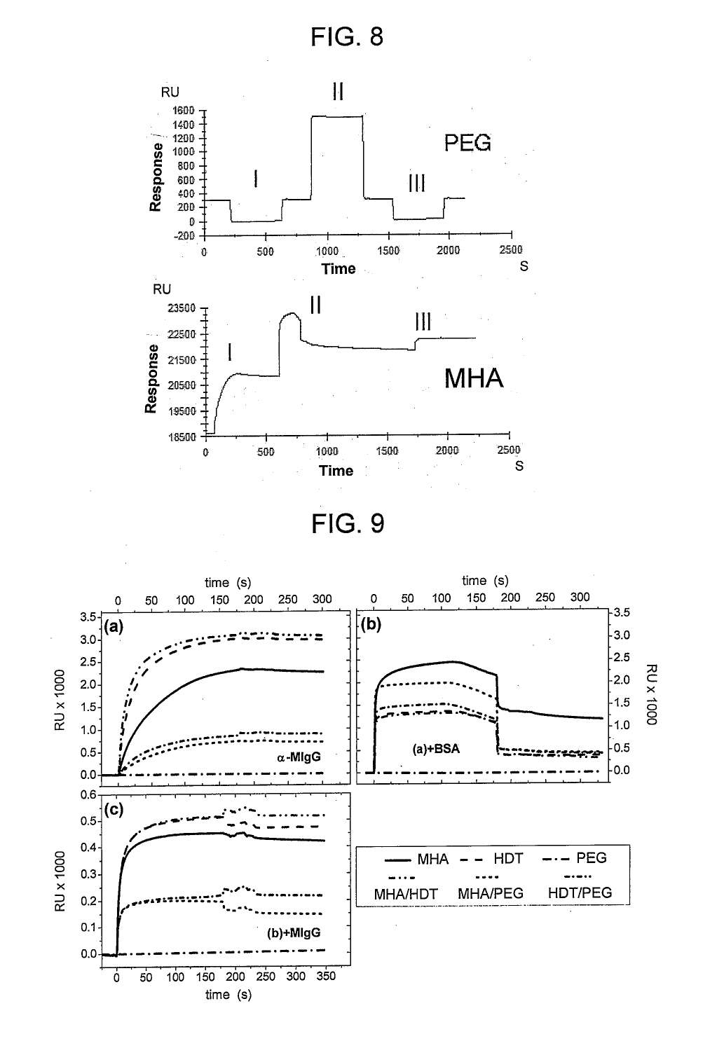

Monoclonal mouse IgG (MIgG) and polyclonal anti-mouse IgG (α-MIgG) were used as model antigen-antibody pair. The sensorgrams were recorded at a constant flow of PBS (pH 7.2) as running buffer, at a flow rate of 20 μl/mL. All protein solutions were prepared in PBS. In a typical experiment, the α-MIgG (37 μg/mL) was first immobilized on the surface either by physisorption or by chemisorption, followed by passivation of the exposed areas by BSA (10 mg/mL) and then exposure to MIgG (50 μg/mL). The differences between physisorption and chemisorption protocols are briefly sketched in FIG. 7. When the chemisorption protocol was used, the surface was first exposed to a freshly prepared NHS/EDC mixture for duration of 12 minutes before flowing the α-MIgG solution. The unreacted esters were destroyed using ethanolamine hydrochloride solution, and then the BSA passivation step was carried out. Use of homo of heterobifunctional cross-linkers that covalently link the antibody to the surface. In our case, the —COOH terminated SAM is treated with NHS/EDC and then the antibody (Amine coupling). The response for each of the pulses was inferred from the change in the RU values (ΔRU) of the stable baselines before injection and 100 s after injection. The antigen binding capacity (ABC) of the surface was calculated from the ratio of the antigen response to antibody response. The surfaces were compared for the areal density of the α-MIgG and the ABC values. FIG. 8 presents separate plots of responses corresponding to the three consecutive steps of the experiment for physisorption on MHA (lower half) and on the PEG-SH SAM (upper half). The latter shows excellent resistance to protein adsorption by exhibiting a zero response to all three steps, while on the MHA surface obviously all consecutive steps lead to an increase in surface coverage. This excellent protein resistance of the PEG-SH SAM is in accordance with what could be expected of oriented high density PEG brushes as confirmed from the XP and IRRA spectra of these SAM (cf. FIG. 5). Please note that the step-like jump in the SPR sensorgram of the PEG-SH SAM is related to the changes in the refractive index of the bulk solution. After rinsing with PBS, the signal remains unaltered with respect to the start value.

Following the same procedure, a number of different nano-patterned as well as non-patterned (“homogenous”) surfaces were analyzed with respect to their antigen binding capacity (ABC). The corresponding sensorgrams are shown in FIG. 9. The homogenous CH₃-SAM and COOH-SAM show significant α-MIgG adsorption, with the hydrophobic CH₃-SAM exhibiting a higher response than the hydrophilic COOH-SAM. Interestingly, the COOH/CH₃ nanopattern yields basically the same response as the homogeneous CH₃ surface, thus giving first evidence for the non-confining character of this pattern. The two nanopatterns with the patches embedded into the PEG matrix, in contrast, show a much weaker response of about 30% of the homogeneous surfaces, thereby indicating Ab adsorption within the fouling patches only. The different surface loading in the first step is well reflected in the subsequent step of BSA passivation (FIG. 9b). Here, CH₃-SAM and COOH/CH₃ nanopattern yield the weakest response of about 11.5% that of the first adsorption step due to their high preload with α-MIgG, while the homogenous COOH surface gives about 52.4% that of the initial α-MIgG adsorption, thereby indicating a higher density of voids on the surface after the first adsorption step. The two nanopatterns using PEG as matrix yield 63.7% for the COOH/PEG pattern and 46.4% for the CH₃/PEG pattern in agreement with the lower adsorption found on the homogenous CH₃ surface as compared to the COOH-SAM.

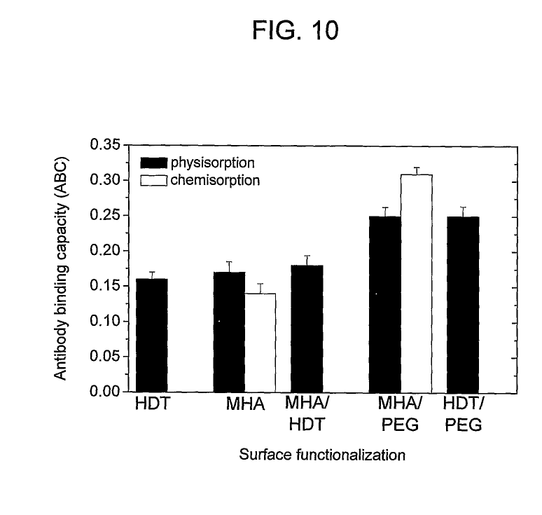

The antigen binding step as shown in FIG. 9c, finally, shows that the surface immobilized α-MIgG is active on homogenous and nanopatterned surfaces. Clearly, the COOH/PEG as well as CH₃/PEG nanopatterned surfaces show a lower response compared to the corresponding homogenous COOH— and CH₃-SAM surfaces. This could be expected when the antibodies are confined to the patches, and surface coverage of the patches is only about 9% of the total area. The COOH/CH₃ nanopatterned surface however shows high response similar to that of the homogenous CH₃-SAM surface, in accordance with the high load of α-MIgG in the first step and thus indicative of the non-confining character of this pattern. A comparison of the activity of immobilized α-MIgG on nanopatterned surfaces (FIG. 10) reveals that the confinement inducing COOH/PEG and CH₃/PEG nanopatterns yield a higher value for the ABC in comparison to the homogenously covered COOH and CH₃ surfaces. Physisorption of α-MIgG onto the COOH nanopatches results in an increase of (47±15.1)%, while physisorption onto the CH₃ nanopatches gives (56±12.3)% in agreement with the higher load of α-MIgG on the corresponding homogenous surface (cf. FIG. 9a). In contrast, the increase of (6±12.5)% on the non-confining COOH/CH₃ nanopatterns is within the experimental error, giving evidence that non-confining nanopatterns do not yield any increase in antibody activity but show the same performance as that of their non-patterned counterparts. Further, the chemisorption protocol for the α-MIgG on the COOH nanopatches results in an even more significant increase in ABC values of the COOH/PEG nanopattern as compared to the homogenous COOH surface, yielding up to (121±22.6)% increase in activity. It is to be noted that all the above-mentioned experiments were performed with the same lot of the α-MIgG antibody to exclude possible lot-to-lot variations in the composition of the polyclonal mixture, which might affect the ABC values. Further, we remind here the fact that the ABC values are independent of the quantity of antibody immobilized on the surface, and are a qualitative indicator of the activity of the immobilized antibodies. The only other way that this enhancement could be observed is through a non-specific binding of the antigen (MIgG) to the surface, which we could exclude from the fact that the PEG layer is sufficiently protein resistant, and also, there being two protein exposure steps (α-MIgG, BSA) preceding the antigen exposure, the non-specific binding sites on surface would have already got occupied. As a further proof of the expected confinement of the antibodies to the MHA patches of the COOH/PEG patterns, AFM tapping mode images were taken on Biacore chips after their removal from the SPR instrument, i.e. after deposition of the full sequence of α-MIgG, BSA, and MIgG molecules. Clearly, as shown in FIG. 4b, the MHA patches, which appeared as ˜8 nm deep depressions in FIG. 4a, are now backfilled with the biomolecules. The line profile (b′) at the bottom of FIG. 4 shows a distribution of heights within the patches with a maximum of 22-24 nm above the PEG matrix. Since there is no evidence for biomolecular adsorption on the PEG, the total height of these “biomolecular pillars” formed on the MHA patches amounts to 28-30 nm. The maximum length of an IgG antibody in the direction of its main symmetry axis is about 14 nm (V. R. Sarma et al., J. Biol. Chem., Vol. 246, pp. 3753-3759, 1971). Thus, the maximum height observed within the patches is in agreement with a situation that both the α-MIgG and the MIgG are oriented with their main symmetry axis perpendicular to the surface. It should be noted that the second layer (MIgG) could specifically adsorb in different orientations, as the α-MIgG used is polyclonal. This hence could give rise to a distribution of heights within the patches, as observed by the AFM. In contrast, COOH/CH₃ nanopatterns as shown in FIGS. 4c/d prior to and after biomolecule adsorption, respectively, do not show any confinement. While some spherical features seem to indicate the presence of Ab/Ag complexes on COOH patches, the biomolecules are randomly distributed across the surface in clots of different size. The unlikely case that only BSA adsorbed on the CH₃ matrix can be excluded because of the high load of this surface with α-MIgG as shown in FIG. 9a. Eventually, as additional confirmation of this complete lack of confinement, the line scan (d′) indicated in FIG. 4d and shown along with line scan (b′) exhibits a lesser height variation than that obtained from the confining nanopattern as is expected for a random deposition of Ab/Ag complexes on the surface.

Our observation of a preferred alignment of the α-MIgG perpendicular to the surface in the case of confining nanopatterns can explain the observed higher ABC values found on such patterns, since the exposure of the complementarity determining regions would be favored in such case. We further presume that the improved antibody density on the MHA patches could have its origins in the freedom for re-orientation experienced by molecules that reach the PEG areas, an enhanced lateral flow of molecules into the fouling patches through diffusion from the surrounding PEG covered areas, or a ‘loading effect’ induced decrease in proportion of denatured molecules within the patches.

### Example 2

### Influence of Nanopatch Size on Activity Enhancement

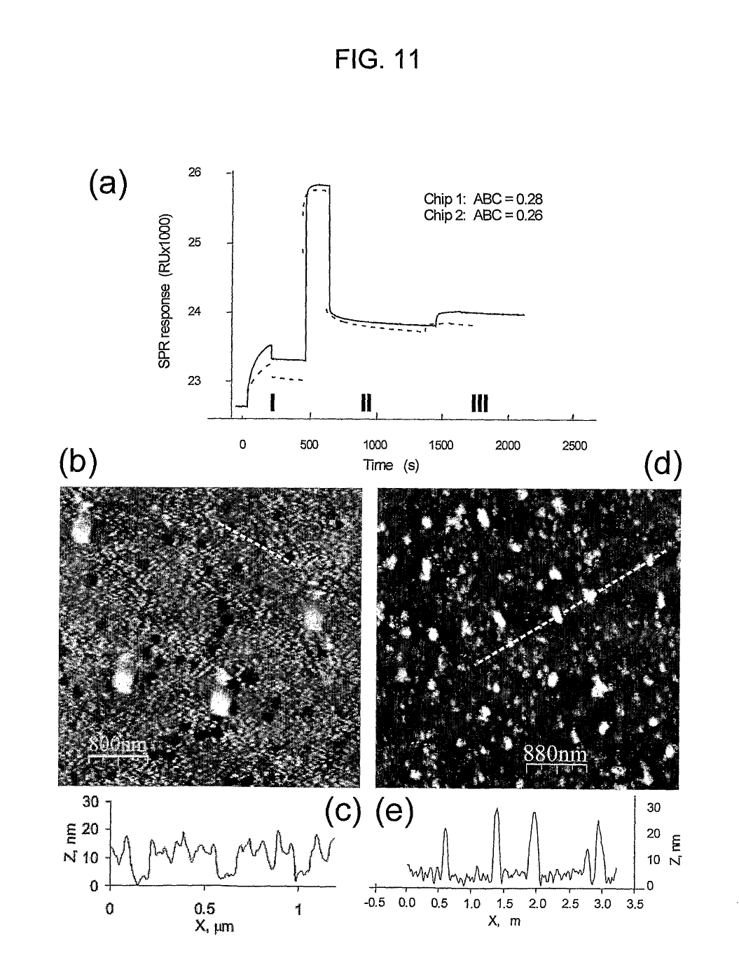

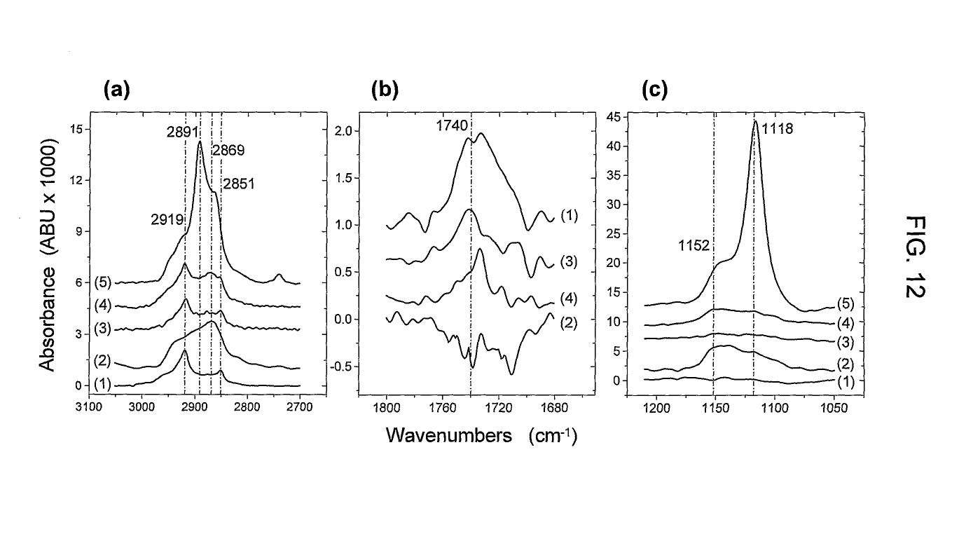

The same experiment as described in example 1 has been performed with a different bead size (all other materials, instruments, and methods same as in example 1). PS beads with a nominal diameter of 200 nm were adsorbed on MHA-coated gold films by means of EDC-mediated adsorption as described above. To achieve random-close-packed monolayers, the amount of EDC had to be slightly adjusted; otherwise the same procedure was followed. After patterning and backfilling of the mask with the non-fouling matrix (PEG-SH SAM), the same Ab/Ag interaction experiment as detailed above was performed, involving polyclonal anti-mouse IgG (37 μg/ml), BSA 1% for blocking of non-specific adsorption sites, and the monoclonal mouse IgG (50 μg/mL) as antigen. FIG. 11(a) displays the SPR sensorgrams for two independently prepared Biacore chips, showing all three steps of the experiment, i.e. antibody physisorption (I), BSA passivation (II), and antigen binding (III). The ABC values obtained were up to 0.28 and 0.26±0.015 on average and thus even higher than those achieved with the 500 nm beads under otherwise same conditions (cf. FIG. 10, results for physisorption on MHA/PEG pattern), which gave 0.25±0.013 on average. By AFM it was verified (FIG. 11(b,c)) that the size of the fouling patches obtained with 200 nm beads as colloidal mask is about 100 nm compared to about 200 nm using the 500 nm beads (cf. FIG. 4). As further confirmed by means of AFM (FIG. 11(d,e)), the Ab/Ag complexes after performing the experiment are confined into these small patches. A reduction in patch size increases the ratio of circumference-to-area of the patches, thereby possibly improving the order and orientation of physisorbed antibodies within the patch due to higher mobility and thus higher potential for reordering of antibodies located near the circumference of the patch. The example given here thus gives some first indication that a reduction in patch size will lead to a further improvement of surface-immobilized antibodies.

### Example 3

### Behavior of Random-Mixed Layers of MHA/PEG

To distinguish the effect of nanopatterning from that of random mixing of two different kinds of molecules on surface, the following control experiment was performed. Randomly mixed monolayers of MHA and PEG (MW ˜2900 Da, Polymer Source, Inc., Montreal, Canada; all other materials, instruments, and methods same as in example 1) molecules were prepared by immersing UV-ozone cleaned gold substrates into mixtures of 50 μM ethanolic solutions of PEG and MHA, respectively, for 3 hours. Two mixture ratios were chosen: (i) 80% PEG solution/20% MHA solution and (ii) 95% PEG/5% MHA to mimic the composition of the nanopatterns of example 1, which were determined to have a fraction of MHA nanopatches of about 10%. It turned out that these ratios were safe lower limits for the MHA fraction of the randomly mixed layers. In an independent experiment, gold wafer pieces were first immersed into the PEG solution for 10-30 min, then into MHA solution for up to three hours. From the study of a similar system it is known that the PEG forms first a coil-like, low density state on surface (Tokumitsu et al., Langmuir, Vol. 18, pp. 8862-8870, 2002), which then could be back-filled with a second molecule. However, when subsequently immersing the PEG-covered samples into MHA solution for up to three hours, it was observed by means of IRRAS that the formerly adsorbed PEG was almost entirely removed from the surface during the MHA adsorption step. Therefore, adsorption from mixed solution was chosen to improve the competition between the two molecules in favor of re-adsorption of PEG. However, under such conditions, it cannot be expected that the mixing ratio of the two molecules on surface resembles that in solution, but instead—as will be shown below—the MHA fraction on surface is higher than the solution fraction. To minimize the deviation from the solution mixture, it seemed to be advisable to keep the immersion time as short as possible. The period of 3 hours was therefore chosen as trade-off between the ability of a homogenous MHA surface to immobilize antibodies to similar extent as after the overnight immersion applied in example 1 (as tested in an independent experiment) and the experimental observation that a certain amount of PEG still remained on surface.

The experimental verification of the formation of mixed films was performed by means of IRRAS. FIG. 12 displays IRRA spectra of MHA (1) and PEG (5) reference samples immersed into the respective solutions overnight, spectra of a PEG sample immersed into solution for 30 min only (2), and those of the two mixed SAMs (PEG/MHA ratio in solution 80/20: (3); 95/05: (4)). Shown are the regions of the CH stretching vibrations (a), which are characteristic for both molecules, the C═O stretching region characteristic to MHA (b), and the C—O—C stretching region (c), which is a fingerprint of the PEG. The pronounced peak at 2919 cm⁻¹ in the spectra of the two mixed SAMs (FIG. 12(a)), which can be assigned to the asymmetric methylene stretch of the MHA, confirms the presence of MHA in the mixtures, in particular in comparison with the spectrum of the 30 min PEG reference sample, which does not exhibit such a pronounced peak in this region. In turn, the peak at 2869 cm⁻¹ is a characteristics of the ether CH₂ stretching vibration and thus confirms the presence of PEG in the mixed SAMs. These findings are corroborated by the spectra of the other two regions. The C═O stretching vibration shows up in the spectra of the MHA reference and the mixed SAMs only. Note that these spectra are close to the noise level of the measurement, not only because of the low number of COOH groups on surface, but also because the region is located within the water absorption bands, which are difficult to eliminate. During acquisition, the spectrum of the 30 min PEG sample was therefore slightly oversampled, so that the water bands appear now as negative peaks in that spectrum, allowing their distinction from other features in this region. The spectra of the C—O—C stretching vibration region, finally, confirm the presence of PEG in the mixed SAMs in an amorphous state. As detailed in example 1 as well as the literature (Tokumitsu et al.), high asymmetry in the intensities of the two peaks at 1118 cm⁻¹ and 1152 cm⁻¹, respectively, is indicative of a highly ordered brush-like state of the molecule as achieved by the PEG reference sample immersed overnight. In contrast, the 30 min PEG reference remained in the coil-like, amorphous state representative for the initial stage of SAM formation in these systems (Tokumitsu et al.). Note, that the crystalline-like state of the PEG was achieved with the nanopatterned samples, where PEG served as non-fouling matrix (example 1). In the mixed SAMs studied here, however, the PEG remains in a low coverage, amorphous state, which indicates that the PEG molecules are too far separated from each other to undergo a coil-to-brush transition by mutual interaction of their chains. Therefore, it can be concluded that the mixed SAMs consist of a sparse layer of PEG with the gaps between the molecules filled with MHA. This is what was intended for comparison with the nanopatterned SAMs.

To investigate into the behavior of these mixed SAMs with respect to antibody immobilization and antibody activity, SPR chips (Biacore SIA kit) were prepared in the same way as the gold-coated silicon wafer pieces used for the IRRAS study by immersing each two chips into the 80/20 and 95/5 mixed solutions, respectively, for 3 hours. Then, the chips were exposed to the same sequence of biomolecules as detailed in example 1, i.e. first adsorption of α-MIgG, followed by passivation of non-specific adsorption sites by means of 1% BSA, and finally, exposure to thus prepared surface to MIgG. The way of performing and evaluating the experiments was identical to that used in example 1. As reference, one SPR chip was immersed into pure MHA for 3 hours. Table 1 shows the results of the total of four SPR chips bearing mixed SAMs for three subsequent steps of antibody immobilization, BSA passivation, and antigen exposure. In Table 1, the values are normalized to the adsorption of the respective biomolecule onto a homogeneous MHA surface. On each of the four SPR chips, two flow channels were measured giving a total of eight experiments. After each step, the change in SPR refractive index units (RU) was determined and normalized to the respective response of the MHA reference chip. Surprisingly, despite of the low coverage with PEG as indicated by the IRRA spectra of FIG. 12, all surfaces show a significant reduction in the amount of biomolecules immobilized on surface. Most of the samples even show non-fouling behavior in the sense that the amount of biomolecules adsorbed is less than 10% that of the reference surface. The variation in the total amount of adsorbed protein reflects the random nature of the mixed films, which might vary in their composition on different locations on the surface. However, there is the trend observable that the chips prepared from the 95/5 mixed solution exhibit a better non-fouling behavior, which is obviously due to the higher amount of PEG on surface (cf. FIG. 12).

In a few cases, desorption instead of adsorption was observed, which might be caused by some loosely bound PEG molecules, which were removed by interaction with incoming biomolecules. In any case, the experiments demonstrate that a random mixture of MHA/PEG acts as a highly protein-resistant coating even at low PEG densities. Therefore, any effect of pinhole defects as they may occur in SAMs of alkanethiolates (Edinger et al., Langmuir, Vol. 9, p. 4-8, 1993) on the results obtained in example 1 can be excluded, in particular in view of the high density brush-like state of the PEG matrix achieved with the nanopatterns. Even in the present study, i.e. with a low lateral density of PEG molecules, the SPR response obtained is more than one order of magnitude smaller than that observed with the nanopatterns, so that any influence of defects in the PEG matrix on the findings of example 1 are negligible even under the unfavorable situation that occasionally a low density PEG matrix should have formed on one of the nanopatterns. Most importantly, the present study illustrates that nanopatterns as prepared in example 1 have distinguished properties and that random mixtures of molecules on surface do not achieve the same performance, in particular with respect to enhancement in biomolecule activity on surface.

TABLE 1

| MHA/PEG ratio | α-MIgG (%) | BSA (%) | MIgG (%) |
|---|---|---|---|
| 20/80 | 13.4 | 36.1 | 22.9 |
| 20/80 | 1.2 | 17.3 | 3.1 |
| 20/80 | 25.9 | −24.3 | 5.2 |
| 20/80 | 0.0 | 2.0 | −2.9 |
| 5/95 | 10.9 | 33.0 | 17.4 |
| 5/95 | 1.9 | 20.0 | 4.4 |
| 5/95 | 0.0 | 0.2 | 0.5 |
| 5/95 | −0.3 | −0.8 | 0.2 |

### Comparative Example 1

### Comparison of Nanopatches Embedded into Fouling and Non-Fouling Matrices, Respectively

In their experiment, Valsesia et al. (Langmuir 2006, 22 (4), 1763-1767), used nanopatterns of MHA/HDT to study the bioactivity of surface-adsorbed antibodies and report an increase of antibody activity of about 4 times that of the respective non-patterned surfaces. The article lacks an experimental section, so that the protocols used are only vaguely known. The most important difference to the work of the present invention is, however, that in the case of MHA/HDT nanopatterns, the antibodies are not confined into the MHA nanopatches, but adsorb on the entire surface. This is becomes clear from the article itself as well as the plurality of work on fouling/non-fouling surfaces that can be found in the literature. FIG. 3 on page 1766 of the article of Valsesia et al. shows clearly that the ELISA experiments performed to determine the amount of antibodies active on surface yield basically the same response for HDT- and MHA-coated surfaces, respectively. Thus, the amount of active antibodies on surface is the same in both cases. A major difference in the total amount of adsorbed antibodies is unlikely due to (i) what is known in the literature for protein adsorption on the two surfaces, (ii) our own findings, and (iii) the AFM images on page 1767, FIG. 4, of the article of Valsesia et al. With regard to the latter, except for a higher density of clods with heights of at least 0.23 nm (the z-scale indicated in the Figure caption) in the image of the MHA-coated surface, the two images appear rather similar, so that no big difference in the total amount of antibodies is expected.

In fact, from the literature it is well known that HDT as a highly non-polar molecule with a high water contact angle (typically above 100 deg) provides a fouling surface, i.e. a surface that promotes the non-specific adsorption of proteins. In many studies on the development of non-fouling surfaces, HDT or similar methyl-terminated aliphatic SAMs serve as a reference system providing an upper limit for potential protein adsorption. These fouling properties of HDT and related molecules have been extensively discussed in the literature (Kingshott, P.; Griesser, H. J. Curr. Opin. Solid State Mater. Sci. 1999, 4, 403-412; Morra, M. J. Biomater. Sci.-Polym. Ed. 2000, 11, 547-569; Leckband, D.; Sheth, S.; Halperin, A. J. Biomater. Sci.-Polym. Ed. 1999, 10, 1125-1147). Wadu-Mesthrige et al. (K. Wadu-Mesthrige et al., Biophysical Journal, Vol. 80, pp. 1891-1899, 2001) fabricated antibody nanopatterns by means of AFM lithography and report explicitly that they had to wash off adsorbed antibodies from the embedding dodecanethiolate matrix to obtain antibody nanopatches on —CHO-terminated surface areas only (page 1896 of said article). Further, our own SPR study gives direct evidence for the similar behavior of HDT- and MHA-coated surfaces, cf. FIGS. 9 and 10. In FIG. 9a, the homogenous HDT film adsorbs even more α-MIgG than the MHA surface used as antibody adsorption patch. As can be easily seen from FIG. 9, the homogenous PEG-SAM is the only surface exhibiting resistance to biomolecule adsorption (with respect to both antibodies, i.e. α-MIgG and MIgG, as well as to BSA 1%).

Altogether, it becomes clear that HDT- and MHA-terminated surfaces behave rather similar in terms of the total amount of antibodies adsorbed on surface as well as their active fraction. Therefore, a nanopattern, where one of these surfaces acts as nanopatch, the second one as matrix is rather unlikely to change anything in this basic behavior. In fact, as we show in FIGS. 9 & 10, the MHA/HDT-nanopatterns did result in the same amount of biomolecules adsorbed as that of their homogenous counterparts and also gave the same fraction of active antibodies on surface (FIG. 10). The AFM analysis of these patterns after biomolecule adsorption showed a random distribution of antibody/antigen complexes on surface (FIG. 4), further suggesting that such fouling nanopatterns embedded into a fouling matrix are unable to confine the antibodies and thus to improve their orientation and thus their activity on surface.

The observation of Valsesia et al. of an enhancement in antibody activity on MHA/HDT patches is therefore in contradiction with our findings. One potential explanation could be that in an ELISA experiment, the solutions used are typically not degassed. In the case of a hexagonally dense-packed MHA pattern embedded into a highly hydrophobic HDT matrix, this might cause a dewetting phenomenon, i.e. the liquids come in contact with the hydrophilic patches only, while the hydrophobic matrix is isolated from the liquid by a thin layer of air that prevents the aqueous solution from an unfavorable interaction with the non-polar surface (see, e.g. Steitz et al., “Nanobubbles and Their Precursor Layer at the Interface of Water Against a Hydrophobic Substrate”, Langmuir 2003; 19(6); 2409-2418). In such case, the biomolecules are selectively adsorbed to the hydrophilic patches, however, not due to the intrinsic properties of the surface but due to selective wetting of one part of the structure only. This is, although an interesting phenomenon, not subject of the present invention (The solutions used in the SPR experiments of the present invention were all well degassed prior to use to avoid such complications).

Heretofore, the present invention is explained with reference to the embodiments. However, various changes or improvements can be applied to the embodiments.

1. A structure for bio-detection comprising:
fouling area and non-fouling area fabricated on a surface of the structure; and an antibody disposed on the fouling area and/or the non-fouling area.

2. The structure according to claim 1, wherein the non-fouling area is a non-fouling matrix, the fouling area is a fouling patch embedded in the non-fouling matrix, the antibody is confined into the fouling patch.

3. The structure according to claim 1, wherein the fouling area and the non-fouling area form a nanopattern.

4. A structure for bio-detection comprising:
a metal surface; and
a small pattern of biomolecules formed on a surface of the metal surface.

5. The structure according to claim 4, wherein the metal surface is a continuous metal surface.

6. The structure according to claim 4, wherein the small pattern is a large-scale nanopattern.

7. The structure according to claim 6, where the large-scale nanopattern is formed by self-assembly methods.

8. A structure for enhancing activity of an antibody comprising:
a nanopattern of fouling and non-fouling areas fabricated on a surface of the structure; and
an antibody confined to the fouling area.

9. A structure for enhancing activity of a biomolecule comprising:
a metal surface; and
a large-scale nanopattern of fouling and non-fouling areas fabricated on the metal surface; and
a biomolecule confined to the fouling area.

10. The structure according to either claim 8, wherein the nanopatterned surface of the structure comprises the sensing surface of a biosensor.

11. The structure according to either claim 8, wherein the nanopatterned surface of the structure is divided into regions with dimensions in the micron or millimeter range.

12. The structure according to claim 11, wherein the different regions of the milli-/micropatterned nanopatterns bear different biomolecules.

13. The structure according to claim 12, where the milli/micropatterned nanopatterns are used for multiplex biosensing.

14. The structures according to claim 8 wherein the nanopatterned surface of the structure is used to promote cell adhesion and cell growth.

15. The structures according to claim 8 wherein the nanopatterned surface of the structure is used to promote growth of biological tissue.

16. The structures according to claim 8 wherein the nanopatterned surface of the structure is used as a surface coating of an implant.

17. The structures according to claim 16, wherein the nanopatterned surface of the structure is used to promote the bio-compatibility of an implant.
